(19) United States
(12) Patent Application Publication
(10) Pub. No.: US 2022/0354940 A1
(43) Pub. Date: Nov. 10, 2022

(54) BEE VACCINES AND METHODS OF USE

(71) Applicant: Dalan Animal Health, Inc., Los Angeles, CA (US)

(72) Inventors: Dalial Freitak, Los Angles, CA (US); Annette Kleiser, Los Angeles, CA (US)

(21) Appl. No.: 17/762,009

(22) PCT Filed: Sep. 17, 2020
(86) PCT No.: PCT/US2020/051301

§ 371 Date: Mar. 18, 2022

Related U.S. Application Data
(60) Provisional application No. 62/901,988, filed on Sep. 18, 2019.

(51) Int. Cl. A61K 39/07

(52) U.S. Cl. CPC A61K 39/07; A61K 2039/552

(57) ABSTRACT

The disclosure provides compositions and methods for treating and vaccinating insects and insect populations from bacterial diseases. The disclosure further provides compositions and methods for prophylactically immunizing honeybee hive to protect from infection with Foulbrood disease. In embodiments, the disclosure further provides compositions and methods for prophylactically immunizing honeybee hive to protect from infection with American foulbrood caused by Paenibaccilus larvae.

### CROSS-REFERENCE TO RELATED APPLICATIONS

This application claims priority under 35 U.S.C. § 119(e) to U.S. provisional application No. 62/901,988, filed Sep. 18, 2019, the content of which is incorporated by reference into this application in its entirety.

### BACKGROUND

Honeybees are used commercially as essential species providing pollination services, as well as honey production. In the United States, commercial beekeepers operate approximately 2.8 million beehives, and honeybees are responsible for pollinating more than one third of all crops including crops such as nuts, berries, fruit, and flowering vegetables. In addition, honeybees also pollinate a variety of wild flowers and, therefore, contribute to the biodiversity of many ecosystems.

Unfortunately, in recent years honeybee colonies have suffered severe declines and colony losses. For example, in the year spanning April 2018-April 2019, over 40% of the 2.8 million commercially operated beehives in the US died; this equates to approximately $700 million in lost revenue to U.S. beekeepers alone, and does not include revenue losses to orchard owners and other farmers due to the lack of pollinating insects.

Chief among the causes of the loss of honeybee colonies are pests and pathogens, including parasites, and viral and bacterial infections.

A particularly devastating bacterial pathogen is American Foulbrood (Afb). American Foulbrood (Afb) is a bacterial disease caused by the spore forming bacterium Paenibacillus larvae (Genersch et al., J. Invertebr. Pathol. 103, (2010b)). The disease can be catastrophic for beekeepers and is challenging to eradicate. The bacterium is present in about 50% of all hives, and the spores can survive up to 70 years. Between 4 and 20% of hives show clinical symptoms at any given time, costing both farmers and beekeepers millions of dollars each year in economic losses.

Unfortunately, there are currently no safe and effective prophylactic solutions available to protect hives from Afb. In acute cases, antibiotics are approved for use for treating bees and may be administered to the hive in the early stages of clinical symptoms to limit spread of the bacterium. However, this is largely ineffective since it is the spores rather than the bacteria that cause the disease. Therefore, burning of the hive is often the only solution to contain the disease.

Thus, there is a need in the art for safe, effective measures for the control and prevention of American foulbrood disease and other related insect disease. This disclosure satisfies this need and provides related advantages as well.

### SUMMARY

The disclosure relates to novel compositions, vaccine formulations and methods for raising an immune response to, and/or vaccinating treating and/or for the prevention foulbrood diseases caused by Paenibacillus sp. including American Foulbrood diseases (AFB) in bees and other insects caused by Paenibacillus larvae, via administration to and/or vaccination of the queen bee with dead non-disease causing Paenibacillus sp., including, but not limited to Paenibacillus alvei (PA) and Paenibacillus dentritiformis (PD). In one aspect, the bees are honeybees.

In contrast to what had been known in the art, Applicant describes administration and/or immunization by applying a stimulus from a non-disease pathogen to treat and/or prophylactically immunize the bees against diseases caused by Paenibacillus sp. such as Afb. Specific match between the disease pathogen and the vaccine strain is not required.

Thus, in one aspect, this disclosure provides a method of protecting insects from disease by applying or administering an immunization stimulus derived from a pathogen other than the disease pathogen to provide broad-spectrum generalized protection.

In an exemplary embodiment, provided herein is a composition containing dead Paenibacillus such as PD and PA in combination or alone for the protection against infection by Paenibacillus larvae (PL). The composition also can contain, for example, bee feed such as honeybee feed.

In other embodiments, the disclosure provides a method for preventing American foulbrood in a population of bees, e.g., honeybees. The method can include administering or feeding to the queen bee a vaccine formulation consisting of dead PB or PA or a combination of both. In one aspect, from about 1.5×10⁶ to about 1.5×10⁸ antigen units/gram of insect food is provided to or fed to the queen bee, worker bees, nurse bees or the larvae.

In one embodiment, provided herein is a composition, comprising, or consisting essentially of, or yet further consisting of, whole cells or cell wall fragments of at least one dead, non-disease causing bacterial species of a bacterial genus, such as dead Paenibacillus sp. such as PD or PA or a combination of both, and a carrier. In one aspect, the composition comprises, or consisting essentially of, or yet further consisting of, whole cells or cell wall fragments of at least two or more, or three or more, of four or more dead, non-disease causing bacterial species of a bacterial genus, such as dead Paenibacillus such as PD or PA or a combination of both, and a carrier. Non-limiting examples of the non-disease causing bacterial species for the composition formulation is a species of the genus Paenibaccilus, Paenibacillus alvei (PA) or Paenibacillus dentritiformis (PD), or a combination thereof. In a further aspect, the one or more, two or more, three or more, or four or more dead Paenibaccilus species is included in the composition, wherein the species is one or more selected from the group consisting of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, and Paenibacillus xylanisolvens, or a combination thereof. In a further aspect, the dead Paenibaccilus species in the composition comprise, or consist essentially of, or consists of Paenibacillus alvei and Paenibacillus dentritiformis, alone or in combination with each other.

Non-limiting examples of a carrier can be a solid or a liquid carrier and can include preservatives, insect nutrients, or other coloring agents as necessary. In one specific embodiment, the carrier is an insect food, such as a queen bee wafer or sugar feed.

In one aspect, the and whole cells or cell wall fragments of the at least one dead, non-disease causing bacterial species of the bacterial genus in the composition are present in a total amount per dose per gram of the composition or food of from about 1.5×10⁶ to about 1.5×10¹¹ antigen units, or from about 1.5×10⁷ to about 1.5×10¹¹ antigen units, or from about 1.5×10⁸ to about 1.5×10¹¹ antigen units, or from about 1.5×10⁸ to about 1.5×10¹⁰ antigen units, or from about 1.5×10⁸ to about 1.5×10⁹ antigen units, or at least 1.5×10⁷ antigen units, or at least about 1.5 x10⁸ antigen units, or at least about 1.5×10⁹ antigen units, or at least about 1.5×10¹⁰ antigen units.

In an exemplary embodiment, provided herein is a composition containing dead Paenibacillus such as PD and PA in combination or individually for the protection of the bee larvae and queen bee progeny against infection by Paenibacillus larvae (PL). The composition also can contain, for example, insect feed such as honeybee feed. The range of whole cells or cell wall fragments of the Paenibacillus are in the ranges provided herein.

In other embodiments, the disclosure provides a method for preventing American foulbrood in a population of bees, e.g., honeybees. The method can include administering to the worker bees, larvae, nursing bees and/or the queen bee a composition as disclosed herein and/or a vaccine formulation comprising, or consisting essentially of, or yet further consisting of dead Paenibacillus such as PD or PA or a combination of both. As is apparent to the skilled artisan, the queen bee may feed on the food or alternatively the worker or nurse bees will ingest the food and feed the queen. In addition, bee larvae may also feed on the composition. The range of whole cells or cell wall fragments of the Paenibacillus are in the ranges provided herein.

In other embodiments, the disclosure provides a treatment for diseases caused by Paenibacillus sp., such as American foulbrood in a population of bees, e.g., honeybees. The method can include administering to the worker bees, nurse bees, larvae and/or the queen bee a composition or formulation comprising, or consisting essentially of, or yet further consisting of dead non-disease causing Paenibacillus such as PD or PA or a combination of both. In one aspect the composition comprises dead PD or PA to treat diseases caused by Paenibacillus larvae, such as Afb. The range of whole cells or cell wall fragments of the Paenibacillus are in the ranges provided herein.

In other embodiments, the disclosure provides a method for preventing American foulbrood in a population of bees, e.g., honeybee larvae or progeny from a queen treated by the method disclosed herein. The method can include administering to the worker bees, nurse bees and/or the mother queen bee a formulation comprising, or consisting essentially of, or yet further consisting of dead non-disease causing Paenibacillus such as PB or PA or a combination of both. The range of whole cells or cell wall fragments of the Paenibacillus are in the ranges provided herein.

Also provided herein is a method of treating or immunizing or raising an immune response in a honeybee larvae against a disease caused by Paenibacillus sp. such as Foulbrood disease known to cause by PL, the method comprising, or alternatively consisting essentially of, or yet further consisting essentially of, administering an effective amount of the composition as described herein to a nurse bees, worker bees or the honeybee queen or honey bee larvae thereby immunizing the queen bee, and the honeybee larvae and progeny produced by the queen bee. In one aspect, the Foulbrood disease is caused by any species belonging to the Family Paenibacillaceae. Without being bound by theory, detection of the antigen that will be passed to the larvae can be detected in the ovaries of the queen bee. This vaccinates the larvae from disease.

The method is useful to protect larvae-queen bees and worker bees from Foulbrood disease that is caused by members of the Family Paenibacillaceae such as PL, PA and PD to provide a population of larvae that has been immunized against Foulbrood via the queen bee that has ingested the vaccine composition.

Further provided herein is a method for treating and/or preventing foulbrood diseases such as Afb in a population of bee, e.g., honeybees comprising, or alternatively consisting essentially of, or yet further consisting of administering to worker bees and the queen bee, a composition as described herein, wherein in one aspect the composition comprises, or consists essentially of, or yet consisting of dead non-disease whole or fragments from the species of Paenibacillus. In another aspect, the dead non-disease species of Paenibacillus are selected from the group consisting of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, and Paenibacillus xylanisolvens, or a combination thereof. In another aspect, the composition comprises one or both of dead non-disease species of Paenibacillus are dead Paenibacillus alvei and Paenibacillus dentritiformis, or a combination of both in combination with other Paenibacillus, or other inactivated cells or fragments of bacteria of the Family Paenibacillace.

In one aspect of this method, the administering comprises feeding the worker bees, nurse bees, larvae and/or the queen bee an suitable amount of wafer of queen candy or sugar feed, wherein the amount administered per dose a vaccine, composition or formulation comprising between about 1.5×10⁶ to about 1.5×10¹¹ antigen units/gram of food, or from about 1.5×10⁷ to about 1.5×10¹¹ antigen units/gram of food, or from about 1.5×10⁸ to about 1.5×10¹¹ antigen units/gram of food, or from about 1.5×10⁸ to about 1.5×10¹⁰ antigen units/gram of food, or from about 1.5×10⁸ to about 1.5×10⁹ antigen units/gram of food, or at least 1.5×10⁷ antigen units/gram of food, or at least about 1.5×10⁸ antigen units/gram of food, or at least about 1.5×10⁹ antigen units/gram of food, or at least about 1.5×10¹⁰ antigen units/gram of food, of the least one dead non-disease species of Paenibacillus or fragments thereof.

Further provided is a method for preparing an insect vaccine and/or composition and/or formulation comprising isolating from at least one, or at least two, or at least three, or at least four or more dead, non-disease causing bacterial species of a bacterial genus, whole cells or cell wall fragments from the least one dead, non-disease causing bacterial species of Paenibaccilus sp.. In one embodiment, the method further comprises admixing the isolated antigen units with an insect food or carrier, such as a queen bee wafer or gel. In one aspect, the non-disease causing bacterial species is a species of the genus Paenibaccilus, e.g., Paenibacillus alvei or Paenibacillus dentritiformis, or a combination thereof. In another aspect, the dead Paenibaccilus species is selected from the group consisting of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, and Paenibacillus xylanisolvens, or a combination thereof. In another aspect, the dead Paenibaccilus species are Paenibacillus alvei and Paenibacillus dentritiformis.

In one aspect, the whole cells or cell wall fragments of at least one, or at least two, or at least three, or at least four or more of dead, non-disease causing bacterial species of a bacterial genus per does is provided between about 1.5×10⁶ to about 1.5×10¹¹ antigen units/gram of food , or about 1.5×10⁷ to about 1.5×10¹¹ antigen units/gram of food, or from about 1.5×10⁸ to about 1.5×10¹¹ antigen units/gram of food, or from about 1.5×10⁸ to about 1.5×10¹⁰ antigen units/gram of food, or from about 1.5×10⁸ to about 1.5×10⁹ antigen units/gram of food, or at least 1.5×10⁷ antigen units/gram of food, or at least about 1.5×10⁸ antigen units/gram of food, or at least about 1.5×10⁹ antigen units/gram of food, or at least about 1.5×10¹⁰ antigen units/gram of food, of the dead non-disease species of Paenibacillus or fragments thereof.

Also provide herein are the treatment and/or vaccine compositions prepared by the methods as described herein as well as the bee or an insect larvae or larvae population from a queen having been immunized by ingestion of the treatment and/or vaccine compositions as described herein. The compositions and/or formulations can be provided in a kit, comprising the composition as described herein and instructions for use.

Other features, objects and advantages of the invention will be apparent from the detailed description which follows.

### BRIEF DESCRIPTION OF THE DRAWING

FIG. 1A, Panels 1-3 and FIG. 1B illustrate the mechanism of vaccination against Afb in honeybees-and is exemplary of vaccination or treatment against other diseases caused by Paenibaccilus sp. Panel 1) Treatment and Vaccination: Worker bees are fed a composition as disclosed herein. Conventional queen feed (queen candy) comprising the composition (the oral vaccine) is taken up by the worker or nurse bees and added to royal jelly in the mandibular glands. The queen bee or the queen larvae is fed the composition (e.g., vaccine) containing royal jelly by the worker and nurse bees. Panel 2) The vaccine is stored in the fat bodies of the larvae and the queen bee and once the queen is fully developed, it is transported to the ovaries of the queen bee. The transfer is mediated by the egg yolk protein Vitellogenin. Panel 3) shows larval treatment. Y axis shows larval survival in %, 72 hours post infection. Queen vaccination with dead American foulbrood results in more resistant larvae then infected with the pathogen (Gray bars, vaccine: 46% larval survival vs. placebo: 23% larva survival, χ2=4.32, p=0.004). No negative effect on larval survival under control conditions with no infection (Black bars, vaccine: 35% larval survival vs. placebo: 32% larva survival, χ2=3.57, p=0.06. FIG. 1B also graphically illustrates the mechanism of vaccination.

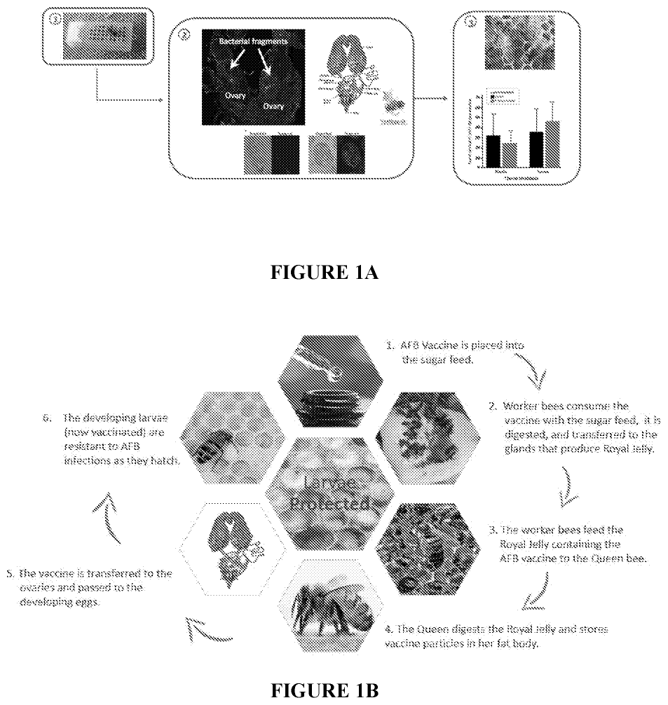

FIG. 2 graphically illustrates an exemplary method to obtain bacterial fragments to be used as antigens. This figure is reproduced from Lodish, H. (ed) Molecular Cell Biology, Sixth Edition, 2008 W.H. Freeman and Company.

FIGS. 3A and 3B show the results of administration and/or vaccination of the honeybee queens by administration of the queen with Paenibacillus denritiformis. The vaccination of honeybees with PD will protect her offspring against infection with PA (FIG. 3A) (survival regression contrast between placebo (light gray) and PD (dark gray) when infected with PA). FIG. 3B shows results of infection with PL (survival regression, contrast between placebo (light gray) and PD (dark gray) when infected with PL. Queen vaccination with dead Paenibacillus sp results in more resistant larvae then infected with the pathogen. (PD vs. PA gray bars±SD, vaccine: 4 approx. 60% larval survival vs. placebo: 40% larval survival, p=0.08) and (PD vs. PL gray bars±SD 35% larval survival vs. placebo 25% larval survival, p=0.18). No negative effect on larval survival under control conditions with no infection (gray bars±SD, vaccine: 15% larval survival vs. placebo: 5% larval survival, p>0.05).

### DETAILED DESCRIPTION

### Definitions

As used herein and in the appended claims, singular articles such as “a” and “an” and “the” and similar referents in the context of describing the elements are to be construed to cover both the singular and the plural, unless otherwise indicated herein or clearly contradicted by context.

As used herein, “about” is understood by persons of ordinary skill in the art and may vary to some extent depending upon the context in which it is used. If there are uses of the term which are not clear to persons of ordinary skill in the art given the context in which the term “about” is used, “about” will mean up to plus or minus 10% of the particular term.

As will be understood by one skilled in the art, for any and all purposes, all ranges disclosed herein also encompass any and all possible subranges and combinations of subranges thereof. Furthermore, as will be understood by one skilled in the art, a range includes each individual member.

The term “exemplary” as used herein refers to “serving as an example, instance, or illustration,” and not “preferred” or “advantageous over other embodiments.”

Unless defined otherwise, technical and scientific terms used herein have the same meaning as commonly understood by a person of ordinary skill in the art. In particular, this disclosure utilizes routine techniques in the field of honeybee husbandry.

The term “American foulbrood” or “American foulbrood disease” or “Afb” as used herein, refers to a fatal bacterial disease of honeybee brood caused by the spore forming bacterium Paenibacillus larvae. Since Paenibacillus larvae causes American fouldbrood disease in honeybees, Paenibacillus larvae is referred to herein as a “disease-causing” bacterium or a “disease species.”

The terms “non-disease causing” species or “non-disease species” as used herein refer to species of bacteria which may or may not be pathogenic but which do not cause the disease being targeted. For example, with respect to American foulbrood disease which is caused by Paenibacillus larvae, exemplary non-disease Paenibacillus species, which may be found for example in the environment, but not on humans or in human wounds may include e.g., Paenibacillus alvei, Paenibacillus dendritiformis Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, and Paenibacillus xylanisolvens. By way of example, Paenibacillus alvei and Paenibacillus dendritiformis are pathogenic and will inflict a brood disease on larvae but the disease is not American Foulbrood caused by Paenibacillus larvae and are thus non-disease forming species.

The term “honey bee” as used herein refers to is any bee which is a member of the genus Apis, primarily distinguished by the production and storage of honey and the construction of perennial, colonial nests from wax. For example, two species of honey bees, namely A. mellifera or A. cerana indica, are often maintained by beekeepers. Honey bees include but are not limited to Apis andreniformis and Apis florea in subgenus Micrapis, Apis dorsata in subgenus Megapis, and Apis cerana, Apis koschevnikovi, Apis mellifera and Apis nigrocincta in subgenus Apis.

The term “bee colony” or “honeybee colony” as used herein, refers to a social unit of bees, e.g., honeybees comprising a colony. The social unit can be of any system organization utilized by bees, which has the purpose of facilitating survival of the group or colony. Typically, a “bee colony” consists of several thousand bees that cooperate in nest building, food collection, and brood rearing. Each member of a “bee colony” has a definite task to perform, and it takes the combined efforts of the entire colony to survive and reproduce. A colony typically comprises a single queen, thousands of workers, and hundreds of drones during late spring and summer. Thus, a bee colony is a “population of honeybees.”

Typically, a “honeybee colony” peaks from late spring to summer and reaches a low point in winter. The social structure of the colony is maintained by the queen and workers and depends on an effective system of communication. Domesticated honeybees are cultivated in “beehives” or “honeybee hives.” Thus, the term “beehive” or “honeybee hive” refers to a structure that functions as a habitation for a colony of bees, e.g., a colony of honeybees.

As used herein, the term “honey bee” is any bee which is a member of the genus Apis. Two species of honey bees, A. mellifer, A. cernan indica, A. andrreniformis, A. florea, A. koschevnikovie, and A. nigrocincta are examples of such.

The term “effective amount” or “an amount effective to” or any grammatically equivalent term or expression refers to the amount that, when administered by any means to a e.g., a honeybee queen, larvae, worker bee or nurse bee or honeybee colony, for treating or preventing a disease or condition, is sufficient to effect treatment or prevention of that disease e.g., foulbrood disease. Typically, an effective dose of antigen for treating and/or immunizing a queen bee and her respective brood is about 1.5×10⁷-1.5×10¹¹ antigen per dose/gram of food and ranges therebetween. In one embodiment, an “effective amount” refers to that amount of a composition which when fed to a queen bee is sufficient to vaccinate the queen and the larvae she produces such that larvae from the vaccinated queen are at least 50%, or at least 45%, or at least 40%, or at least 35%, or at least 30%, or at least 25%, or at least 20%, or at least 15%, or alternatively at least 10%, or at least 5%, more resistant against infection with American foulbrood than are larvae from an unvaccinated queen. In order to measure it the following protocol will be followed. Depending on the queen provider queens will be placed to the queen cages with wafers containing the antigen. Each queen will be provided with 6-10 worker bees and kept in the lab under ambient temperature of about 23-26 C in the dark for 8 days. The survival will be recorder daily. After 8 days, the queens will be inserted into recipient hives (which will be prior checked for the queen cells—removed in the case some are found) in their cages, opening the door to the cages (depending on the design of the cage) allowing the access to the hive. All the hives will be allowed to acclimate for 5 days, prior to checking, if the queens are released. After that the presence or absence of the queens and eggs will be recorded. 10 days after queen placement, all the hives will be monitored for the presence of the eggs. In the case of the suitable egg laying activity (about 60-100 eggs per hive), the infection experiment to study the vaccine efficacy can be carried out 2 weeks after placing the queens in the hives.

From each hive 15-30 larvae (L1) per hive will be crafted in the laboratory conditions and exposed to virulent spores for 48 hours and then transferred to new larval food with no spores. The spores will be mixed into the larval food. Which consists of Royal jelly 50%, Glucose 6%, Fructose 6%, Yeast extract 1%, Distilled water 37%. New food will be given each 2 days (day 2=700 μl, day 4=800 μl, day 6=1000 μl, day 7=1000 μl, day 8=end of monitoring) and the survival will be monitored on the daily bases; there as dead larvae will be removed during the observation. Larvae will be crafted into 379 microliter droplet (day 0) of larval food and 5 larvae per droplet will be placed. Parallel group with no spores in the food will serve as an environmental control. Based on the survival of the larvae in different groups the vaccine efficacy will be calculated. [Please advise how this will be measured.]

The term “nurse bee” as used herein intends are the bees that feed the worker larvae worker jelly which is secreted from glands that produce royal jelly.

The term “worker bee” as used herein intends any female (eusocial) bee that lacks the full reproductive capacity of the colony's queen bee.

The term “brood” intends the three developmental stages in bees, which are collectively known as brood. Bees begin in eggs, which hatch to become larvae (plural). The larvae is legless and is specialized to eat.

The term “prophylactic” or “vaccine” refers to an agent that acts to prevent a disease e.g., a honeybee disease, such as e.g., Foulbrood caused by the bacterium Paenibacillus larvae in the brood.

The term “vaccinate” as used herein, refers to means for producing immunity against a disease e.g., producing immunity to Paenibacillus larvae, so as to prevent a disease or condition from occurring (prophylactic treatment) or inhibiting the disease from spreading (slowing or arresting its development) in the brood, larvae, progeny or colony.

The term “treatment” intends to raise an immune response in the queen which is then passed on to her progeny.

The term “raise an immune response” intends that the treatment the vaccine or treatment produces the non-disease causing antigens in the ovaries of the honey bee queen or to the developing eggs.

An “antigen/unit” intends the number of cells or antigenic fragments of the non-disease pathogen.

The term “dose” intends the amount provided to the queen bee, the worker bee, nurse bee or larvae in one feeding or unit amount of food.

### Modes for Carrying Out the Disclosure

Honeybees, in particular Apis mellifera, are the primary pollinators of most commercial crops in North America, and are the most actively managed pollinators in the world. Accordingly, honeybees have a significant economic impact, and therefore, maintaining healthy bee colonies is an essential aspect of much agricultural practice.

Recently, commercial honeybee colonies have suffered extensive losses due to diseases such as brood disease including American Foulbrood (Afb) caused by the bacterium Paenibacillus larvae but also brood disease caused by PA and PD which resemble Afb in symptoms.

American Foulbrood (Afb) is a catastrophic disease of bee colonies which is both contagious and extremely challenging to treat. It can wipe out entire colonies. Often, the most effective treatment for Afb is simply to burn all hives and equipment. Fortunately, the present disclosure provides novel compositions and methods for treating and/or vaccinating honeybee queens and their progeny again American foulbrood and thereby, prophylactically immunizing honeybee colonies to prevent disease.

It has previously been described that immunization of bees against Afb can be accomplished by inoculating queen bees with dead bacteria of the same species, e.g., PL to immunize against PL-causing disease such as Afb (see e.g., U.S. Patent Application Publication 2018/021529). Applicant is the first to disclose that other non-disease causing bacterial species can be used to prepare a vaccine to prophylactically treat and/or immunize the bees against American foulbrood. Furthermore, this protection applies to various brood diseases where vaccination with one Paenibacillus strain both pathogenic and non-pathogenic can protect against infection with another Paenibacillus strain. Applicant is the first to disclose that specific match between the disease pathogen and the vaccine strain is not required. This distinguished this disclosure from the prior art where such in neither contemplated nor disclosed.

It is unexpected that non-disease causing bacteria would defend the host organism against disease. The disease causing pathogens have highly specific virulence factors, for example Cry-toxins in the Bacillus thyringiensis. This bacterium is entomopathogenic bacterium infecting different insect groups, however, the infectivity depends on the type of the toxin produced. For example Cry-2A infects Lepidopteran hosts only, Cry4B infects mosquitoes and mCry-3A infects beetles. This high host specificity even within the same bacterial species, makes the discovery of this disclosure, that we can protect a host against a disease with vaccination with non-disease causing bacterium is unexpected and novel.

Thus, one aspect of the disclosure is a method of treating and/or protecting insects from diseases by applying an treatment and/or immunization stimulus derived from a species of Paenibacillus which does not cause the disease and thereby offering broad-spectrum generalized protection. Non-limiting examples of insects that can be protected using the vaccine formulations disclosed herein include e.g., bees, honeybees (Apis sp.), bumblebees (Bombus sp.) which can be protected from infection and disease caused by e.g., Paenibacillus larvae the causative agent of American foulbrood. As used herein, the term “applying an immunization stimulus” means to prevent disease from disease-causing Paenibacillus sp.. The composition or formulation or vaccine can be administered to the bees or larvae one or several times a year, e.g., one, two, three, for or more times per year before or after hibernation. It can be administered as a single dose or as several doses.

The term “genus” as used herein has its customary meaning as known in the art. In general, genus is defined as taxonomic rank used in the biological classification of living organisms, in the hierarchy of biological classification, genus comes above species and below family. By way of example, the Paenibacillus genus is of facultative anaerobic, endospore-forming bacteria, classified by Ash et al. 1994 (see e.g., Ash, C., Priest, F. G. & Collins, M. D. (1994). Paenibacillus gen. nov. and Paenibacillus polymyxa comb. nov. In Validation of the Publication of New Names and New Combinations Previously Effectively Published Outside the IJSB, List no. 51. Int J Syst Bacteriol 44, 852). Disease pathogen is an infective biological agent, that causes an illness in a host, characterized by certain features such disturbing the function of an organism, and seriously impairs host, including its demise. Broad spectrum generalized protection is achieved when immunization with a bacteria of specific genus or a multiple species of the genus protects against infection of the host then encountering a disease causing pathogen that was not included in vaccine preparation.

In exemplary embodiments, the disclosure provides a novel vaccine composition and/or formulation and methods for the treatment and/or prevention American Foulbrood diseases (AFB) in honeybees, via vaccination of queen bees with dead Paenibacillus sp. that are not Paenibacillus larvae e.g., Paenibacillus alvei (PA) and Paenibacillus dentritiformis (PD).

### Preparation of a Composition for Preventing Bacterial Disease in an Insect Population General Methods

This disclosure utilizes routine techniques in the field of honeybee husbandry. Basic texts disclosing terms and methods in honeybee husbandry include e.g., Queen Rearing and Bee Breeding Harry H. Laidlaw and Robert E. Page (1997). The below methods are intended to be exemplary only, and can be applied more broadly to other insects, diseases and bacteria.

### Preparation of Oral Vaccine

In embodiments, the disclosure provides compositions comprising organisms of a specific phenotype. The composition protects against diseases caused not only by species in vaccine preparation, but also against similar diseases caused by other species in the same genus. Such species that can protect against the disease caused by another species is called a non-disease organism.

For example, all Paenibacillus species cause brood disease, that look very similar in their appearance. However, only Paenibacillus larvae infections cause American Foulbrood. Fortunately, the disclosure provides for the ability to protect against Paenibacillus larvae infections as well as against other bacterial brood infections. Therefore, the term “disease-causing” and/or “non-disease causing” are relative terms that relates to the targeted disease. For example, with reference to American Foulbrood, wherein the causative agent is Paenibacillus larvae, Paenibacillus larvae is the “disease causing” bacterium. In this context, other Paenibacillus sp., e.g., PA and/or PD, are “non-disease causing”.

In embodiments, vaccine compositions comprising non-disease organisms are prepared as disclosed herein in Example 1. Briefly, non-disease causing bacteria are grown on liquid or solid media (agar plates, fermenters, flasks) and collected by methods known in the art. Whereas both liquid or solid media can be plant or animal origin.

Non-limiting example for the inactivation of the collected bacteria are the use of formaline, binary ethylenimine (BEI) or heat and pressure (autoclaving). Any method known in the art for killing bacteria may be used. Following killing, the cells can be lysed and large fragments of separated by centrifugation for between 1-4 hours (or longer, depending on the volume, size and concentration) at a speed from about 10,000 g-to about 60,000 g. The supernatant is recovered and further fractionated as disclosed in Example 1. Alternatively whole cell preparations can be used.

[66] The supernatant can be freeze dried, lyophilized or in solution and can be administered to the bees in different forms, such as e.g., feed, spray, injection. One mode of administration, includes but is not limited to a preparation of the queen candy, which can be purchased prepacked at beekeeping supply stores or prepared by methods known in the art.

In an exemplary embodiment, the composition and/or vaccine preparation is given to worker bees that will incorporate the vaccine with royal jelly in the mandibular glands and feed it to the queen bee or it is given directly to a queen bee to effectively treat or immunize the queen and her resulting brood of larvae against the pathogenic bacterial disease (e.g. against American foulbrood), for example a vaccine preparation with Paenibacillus can protect against American Foulbrood from 3-12 months.

### Using the Compositions

The oral vaccine technology disclosed herein, is administered via the queen bee feed (“queen candy”), any other bee feed or supplement that is taken up by the queen, nurse or the worker bees and fed via royal jelly to the queen bee or queen bee larvae. It is based on the concept of trans-generational immune priming (see e.g., Salmela H, Amdam G V, Freitak D (2015) Transfer of Immunity from Mother to Offspring Is Mediated via Egg-Yolk Protein Vitellogenin. PLoS Pathog 11(7):e1005015.https://doi.org/10.1371/journal.ppat.1005015.), which immunizes the queen bee, and in turn transfers the antigen and the acquired innate immune signal to the eggs and thus protecting newly hatched larvae and bees against harmful pathogens.

As is known in the art, insect immune systems can recognize specific pathogens and prime offspring immunity. High specificity of immune priming can be achieved when insect females transfer immune elicitors into developing oocytes. The molecular mechanism behind this transfer occurs through the egg-yolk protein vitellogenin. Vitellogenin binds to bacteria or fragments of bacteria which comprise cell wall, and recognizes pathogen-associated molecular patterns to transmit immune-priming signals (see e.g., Salmela H, Amdam G V, Freitak D (2015) supra).

Without being bound by theory it is believed that certain cell wall markers, such as within a bacterial genus are shared Paenibacillus sp.. Exposure to these cell wall markers, either as part of whole cells or cell wall fragments, stimulates immunity to other members of the genus, not just to the species from which the cell wall markers are derived. Thus, exposure to the cell wall markers from one species of the genus e.g., a non-disease causing species, stimulates immunity to other members of the genus, including disease-causing (pathogenic) members of the genus. Thus, in exemplary embodiments, immunizing with compositions comprising at least P. alvei or P. larvae protects against the infection with P. alvei and also against infection with P. larvae.

In one embodiment, but not limited to the composition of oral vaccine is delivered as part of the normal husbandry practice for queen bees. The queens together with 8-10 worker bees are placed into ‘queen cages’ supplied with sufficient feed (queen candy) to last up to one or two weeks. The oral vaccine is added to the queen candy, on which the queen bee and the worker bees will be feeding for 3-7 days after which she is placed into the new hive, ready to lay eggs and create the new protected bee colony.

In another embodiment the vaccine is placed into the queen candy in the queen shipping box or into the nuc or queen rearing hive where nurse bees will consume the vaccine with the queen candy and transport it to their royal jelly glands where it is mixed with royal jelly that is fed to the queen bee larvae or the queen bee.

In another embodiment the vaccine is fed to the developing queen larvae during the larval rearing phase in the queen rearing hives.

### EXAMPLES

### Example 1

The following Example illustrates an exemplary method for preparing a composition comprising an effective amount of more than one species of dead, fragmented Paenibaccilus.

Paenibacillus alvei (PA) and Paenibacillus dentritiformis (PD) (or other antigens) are prepared to provide a vaccine or treatment for honeybee queens. The method disclosed below provides for uniformity across all vaccine preparations.

Seed cultures are innoculated from frozen glycerol stocks of Paenibacillus alvei (PA) or Paenibacillus dentritiformis (PD) or other Paenibacillus sp. The inoculated plates are grown at 35° C. in a darkened growth chamber for 4-12 days.

Paenibacillus colonies are harvested by washing the plates with 5 mL ice-cold H₂O and scraping them off into a falcon tube (or glass bottle). The optical density and 600 nm (OD600) is measured using standard methods. Equal volumes of harvested bacteria culture and H₂O are mixed in two replicates into cuvettes. Based on the OD600 reading, antigen solution was prepared to the desired concentration—at least 1.5×10⁹ bacterial cells/mL. Bacterial solution is autoclaved or 15 min at 121° C. to kill the bacteria.

### Method of Obtaining Bacterial Fragments to be Used as Antigen

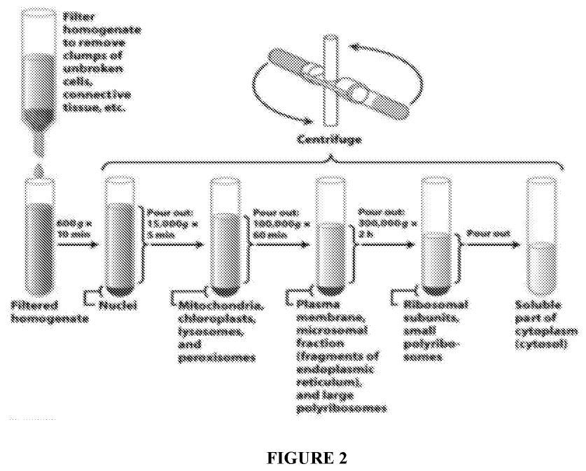

FIG. 2 graphically illustrates an exemplary method to obtain bacterial fragments to be used as antigens. BugBuster® can be used on fresh or frozen cell pellets. Cells are harvested from liquid culture by centrifugation at 10,000×g for 10 min using a weighed centrifuge tube. For small scale extractions (1.5 ml or less), centrifugation can be performed in a 1.5-ml tube at 14,000-16,000×g. Liquid is decanted and the pellet is allowed to drain, removing as much liquid as possible. Pellet is weighed. Once cells have been harvested, but not autoclaved, they can be pelleted and stored at +4° C. The cell pellet is resuspended at room temperature with BugBuster® Master Mix by pipetting or gentle vortexing, using 5 ml reagent per gram of wet cell paste. This typically corresponds to about 2.5 ml per 50-ml culture. The cell suspension is incubated on a shaking platform or rotating mixer at a slow setting for 10-20 min at room temperature. Incubated for 1 h, shaking, +25° C. Insoluble cell debris is removed by centrifugation at 16,000×g for 20 min at 4° C. The supernatant is transferred to a fresh tube. Clarified extracts are maintained on ice for short term storage (2-3 h) or frozen at −20° C. until needed. Cell homogenate is transferred into centrifuge tubes. Centrifugation is done according to the scheme below, supernatant is transferred to new centrifuge tube.

### Centrifugation Steps Used (at 4 C):

1) 10 min 800×g
2) 10 min 1500×g
3) 60 min 10000×g
4) 3 h 20000×g

add 500 μl PBS to each pellet and vortex.

Collected fragments are used as an antigen in the wafer to vaccinate or treat the queens and their progeny.

### Example 2

The following Example illustrates an exemplary method for immunizing a honeybee hive against American foulbrood disease using non-disease disease causing Paenibacillus.

One wafer comprising a composition of non-disease causing Paenibacillus is used to vaccinate or treat at least 7 queens. The wafer comprises contains 80 g queen candy (e.g., commercial bee feed—Nordzucker), 1 mL antigen containing 1.5×10⁸-1.5×10¹⁰ antigen in 1× Phosphate buffered saline (PBS; 1× dilution, pH 6.6-7.2, 137 mM NaCl, 2.7 mM KCl, 9.5 mM Phosphate buffer). The concentration of the antigen is measured photometrically, with as OD600.

The unit for treatment or vaccination is the honeybee queen, before placement into a hive. As is well known in the art, to introduce a queen bee into a hive, the queen is first placed alone in a queen cage and fed queen candy. In this Example, about one week prior to being placed in a hive, queens are fed commercial bee feed wafers that comprise the composition of non-disease causing Paenibacillus antigen(s) prepared as disclosed herein in Example 1.

After feeding the queen bee the queen candy wafer comprising the non-disease causing Paenibacillus antigens, the queen bee is placed in the hive.

After placement into a hive, queens start laying eggs within 3-5 days and continue until the end of the brood production period. The larvae laid by the queen will be at least 30% more resistant against infection with American fouldbrood (Paenibacillus larvae).

Resistance against infection is determined by grafting freshly hatched (12-36 hours) honeybee larvae from both vaccinated and placebo treated hives into the artificial rearing conditions in the lab. All the larvae are then subjected either to oral infection treatment, consisting of food inoculated with living P. larvae spores or will be subjected to control treatment (food with no spores). The survival will be monitored for 8 days and the resistance to infection measured by comparing the survival of immunized vs non immunized larvae.

### Example 3

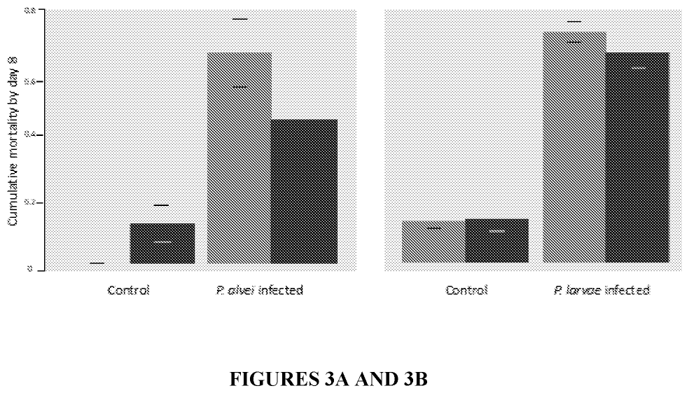

FIGS. 3A and 3B show the results of administration and/or vaccination of the honeybee queens by administration of the queen with Paenibacillus denritiformis. For the testing the antigen PD against the PL infection, one queen was vaccinated with 1.5×10⁸ CFU of the antigen in the 8 grams of the bee feed and one queen served as placebo control, with no antigen in the 8 grams of bee feed. Queens were kept in the queen cages (standard) with 8 worker bees at the room temperature (approx. 23° C.) and of relative humidity of 60% for 8 days. After that they were placed into the donor hives and allowed to lay eggs. 3 weeks after placing the queens in the hives, L1 larvae were grafted in the lab and infected with spores of P. larvae, larval food with no spores served as environmental control. 15 larvae per queen per treatment was grafted into different treatments and monitored in the lab for their survival for 8 days. For testing the antigen PD against PA, similar protocol was followed as described earlier, with exception of using 3 Placebo hives and 1 hive for antigen PD, all the hives were sampled 3 times. Efficacy: Queen vaccination with dead Paenibacillus sp results in more resistant larvae then infected with the pathogen (PD vs. PA gray bars, vaccine: 4 approx. 60% larval survival vs. placebo: 40% larva survival, p=0.08) and (PD vs. PL gray bars 35% larval survival vs. placebo 25% larval survival, p=0.18). No negative effect on larval survival under control conditions with no infection (gray bars, vaccine: 15% larval survival vs. placebo: 15% larval survival, p>0.05).

Mortality of the infected larvae was monitored until day 8 during the experiment. Day 8 is a crucial time point in the development of the honey bee, as it marks the pupation event. The laboratory handling of honey bee pupae is very cumbersome, as they are surrounded with very thin cuticle, which breaks on even very gentle touch. Hence, monitoring the effect of the infection upon pupation event was terminated. Further, the Foulbrood diseases kill the bee larvae by day 5, and therefore, once the bee larvae pupate on day 8 it has survived the infection and it makes the monitoring for the survival of infection obsolete.

### Embodiments

In one aspect, provided herein is a composition comprising, or consisting essentially of, or yet further consisting of, whole cells or cell wall fragments of at least one dead, non-disease causing bacterial species of a bacterial genus and optionally a carrier. In one aspect, the carrier is an insect food. In one aspect, the non-disease causing bacterial species comprises the genus Paenibaccillus, non-limiting examples of such include Paenibacillus alvei or Paenibacillus dentritiformis, or a combination thereof. In another aspect, the one or more dead, non-disease causing bacterial species is selected from the group consisting of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus maceran-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, and Paenibacillus xylanisolvens, or a combination thereof. In a further aspect, the dead, non-disease causing bacterial species comprises, or consists essentially of, or yet further consists of Paenibacillus alvei and Paenibacillus dentritiformis.

In one embodiment, the compositions as disclosed herein comprises, or consists essentially of, or yet further consists of from about 1.5×10⁶ or 1.5×10⁷ to about 1.5×10¹¹ antigen units of whole cells or cell wall fragments of the dead non-disease causing species per gram of carrier, e.g. insect food.

In one embodiment, a method of one or more of: treating, vaccinating immunizing a bee queen or bee larvae against Foulbrood disease is provided. In one aspect the bee queen or larvae is a honeybee. Other bee species are described herein. The method comprises, or consists essentially of, or yet further consist of administering an effective amount of the composition as disclosed herein to one or more of worker bees, nurse bees, larvae or the bee queen, e.g. honeybee queen, thereby immunizing the queen bee or the honeybee larvae produced by the queen bee. In one embodiment, the Foulbrood disease is caused by a species of Paenibacillus. In one aspect the Foulbrood disease is caused by P. larvae. In another embodiment, the Foulbrood disease is American Foulbrood. In a further aspect, the Foulbrood is caused by P. larvae and the disease is American Foulbrood. In a yet further embodiment, the composition administered comprises or consists essentially of one or more of dead whole cells or cell wall fragments of dead, non-disease causing Paenibacillus, e.g., Paenibacillus alvei and Paenibacillus dentritiformis and the Foulbrood disease is American Foulbrood disease. For these embodiments, the queen bee, worker bee, nurse bee or the larvae are fed or administered from about 1.5×10⁶ or 1.5 x10⁷ to about 1.5×10¹¹ antigen units of whole cells or cell wall fragments per dose. In another embodiment, the queen bee, worker bee, nurse bee or larvae are administered or fed at least one, or at least two, or at least three, or at least four, or at least five, or at least six, or at least seven, or at least eight, or at least nine, or at least 10 dose(s) of from 1.5×10⁷ to about 1.5×10¹¹ antigen units of whole cells or cell wall fragments.

In another embodiment, provided herein is a method for preventing American foulbrood in a population of bees or bee progeny comprising, or consisting essentially of, or yet further consisting of, administering or feeding to a queen bee, worker bees, nurse bees or larvae a composition comprising, or consisting essentially of, or yet further consisting of, whole cells or cell wall fragments of at least one dead, non-disease species of Paenibacillus. In one aspect of this method, the American Foulbrood is caused by P. larvae. In one aspect, the queen bee and bees are honeybees. The queen bee can feed directly on the composition or it can be fed to her by the worker or nurse bees. Alternatively or in addition, the larvae feed on the composition of this disclosure.

In one aspect, the disclosure provides a method for preventing American foulbrood in a population of honeybees or honeybee progeny comprising, or consisting essentially of, or yet further consisting of, administering or feeding to a honeybee queen bee from about 1.5×10⁶ or 1.5×10⁷ to about 1.5×10¹¹ antigen units of whole cells or cell wall fragments of PA or PD, or both per gram of food or dose. In one aspect, the queen is fed from about 1.5×10⁷ to about 1.5×10¹¹ antigen units per gram of food or dose. The dose can be administered in queen bee food which the honeybee queen will eat thereby protecting the larvae and her progeny.

In one aspect of this embodiment, the dead non-disease species of Paenibacillus that is administered or fed are one or more of the Paenibacillus is selected from the group consisting of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, and Paenibacillus xylanisolvens, or a combination thereof. In a particular embodiment, the dead non-disease species of Paenibacillus are selected from dead Paenibacillus alvei, Paenibacillus dentritiformis, or a combination thereof are administered to the queen bee, nurse bees or worker bees. In one embodiment of this method, the queen bee is feed queen feed comprising, or consisting essentially of, or yet further consisting of, from about 1.5×10⁶ or 1.5×10⁷ to about 1.5×10¹¹ antigen units per dose of at least one dead non-disease species of Paenibacillus or fragments thereof. In a further aspect, the composition administered or fed to one or more of the queen bee, worker bees, nurse bees or larvae comprises or consists essentially of one or more of dead Paenibacillus alvei and Paenibacillus dentritiformis whole cells or cell wall fragments. In a further aspect, the one or more of the queen bee, worker bees, nurse bees or the larvae are administered from about 1.5 x10⁷ to about 1.5×10¹¹ antigen units of whole cells or cell wall fragments per dose of the dead, non-disease causing Paenibacillus.

In the methods the one or more of the queen bee, worker bees, nurse bees or larvae are administered or fed at least one, or at least two, or at least three, or at least four, or at least five, or at least six, or at least seven, or at least eight, or at least nine, or at least 10 dose(s) of from 1.5×10⁷ to about 1.5×10¹¹ antigen units of whole cells or cell wall fragments.

[95] In one embodiment of this disclosure, provided is a method for preparing an insect composition or vaccine comprising isolating whole cells and/or cell wall fragments from at least one dead, non-disease causing bacterial species of a bacterial Paenibaccilus and then optionally, comprising admixing the isolated antigen units with an insect food. In one aspect, the insect food is selected from a queen bee wafer, a sugar eater, a spirulina supplement, queen candy in the shipping container, a queen cell, an insect supplement. In a further aspect, the non-disease causing bacterial species is a species of the genus Paenibaccilus, e.g., Paenibacillus alvei or Paenibacillus dentritiformis, or a combination thereof. Alternatively, the one or more of dead Paenibaccilus species is selected from the group consisting of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus maceran-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, and Paenibacillus xylanisolvens, or a combination thereof. In a specific embodiment, the dead Paenibaccilus species are Paenibacillus alvei or Paenibacillus dentritiformis or a combination thereof.

In a further aspect, the method further comprises formulating the isolated whole cells or cell wall fragments to provide from about 1.5×10⁶ or 1.5×10⁷ to about 1.5×10¹¹ antigen units per dose of insect food. In a further aspect, provided herein is a composition prepared by the methods as disclosed herein. In one aspect, the composition is a vaccine composition.

In a further aspect, kits are provided that contain the composition or vaccine as described herein and optionally instructions for use.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A composition comprising whole cells or cell wall fragments of at least one dead, non-disease causing bacterial species of a bacterial genus and a carrier.

2. (canceled)

3. The composition of claim 1, wherein the non-disease causing bacterial species comprises the genus Paenibaccillus.

4. The composition of claim 1, wherein the dead, non-disease causing bacterial species is one or more of selected from the group consisting of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, and Paenibacillus xylanisolvens, or a combination thereof.

5. The composition of claim 1, wherein the composition comprises dead, non-disease causing bacterial species Paenibacillus alvei and Paenibacillus dentritiformis.

6. The composition of claim 1, wherein the composition comprises from about 1.5×10⁷ to about 1.5×10¹¹ antigen units of whole cells or cell wall fragments per gram of insect food or carrier.

7. A method for one or more of: vaccinating, treating or immunizing a bee queen or bee larvae against Foulbrood disease, comprising administering an effective amount of the composition of claim 1 to a bee selected from one or more of worker bees or nurse bees that feed the bee queen, bee larvae or the bee queen, thereby immunizing the queen bee or the bee larvae produced by the queen bee.

8. The method of claim 7, wherein the Foulbrood disease is caused by a species of Paenibacillus.

9. The method of claim 8, wherein the Foulbrood disease is American Foulbrood disease.

10. The method of claim 7, wherein the composition comprises or consists essentially of one or both of Paenibacillus alvei or Paenibacillus dentritiformis.

11.-13. (canceled)

14. A method for preventing or vaccinating against American foulbrood in a population of bees, optionally honeybees, comprising administering to a bee queen, a composition comprising whole cells or cell wall fragments of at least one dead, non-disease species of Paenibacillus.

15. The method of claim 14, wherein the dead non-disease species of Paenibacillus are selected from one or more from the group consisting of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, and Paenibacillus xylanisolvens, or a combination thereof.

16. The method of claim 14, wherein the dead non-disease species of Paenibacillus are dead Paenibacillus alvei or Paenibacillus dentritiformis, or a combination thereof.

17.-22. (canceled)

23. A method for preparing an insect composition or vaccine comprising isolating whole cells or cell wall fragments from at least one dead, non-disease causing bacterial species of a bacterial Paenibaccilus.

24. The method of claim 23, further comprising admixing the isolated antigen units with an insect food.

25.-29. (canceled)

30. A composition or vaccine composition prepared by a method of claim 24.

31. (canceled)

32. A kit comprising the composition of claim 1, and instructions for use.
